(19) United States
(12) Patent Application Publication
(10) Pub. No.: US 2010/0160921 A1
(43) Pub. Date: Jun. 24, 2010

(54) CANCELLOUS BONE DISPLACEMENT SYSTEM AND METHODS OF USE

(75) Inventors: Benjamin K. Sun, San Francisco, CA (US); J. Kevin McGraw, Dublin, OH (US); James L. Pacek, Lakeway, TX (US); Jennifer George, San Jose, CA (US)

Correspondence Address: ARTHROCARE CORPORATION;ATTN: Matthew Scheele, 7500 Rialto Boulevard, Building Two, Suite 100, Austin, TX, 78735-8532, US

(73) Assignee: ArthroCare Corporation, Austin, TX (US)

(21) Appl. No.: 12/339,578

(22) Filed: Dec. 19, 2008

(51) Int. Cl. A61B 17/58; A61B 19/00

(52) U.S. Cl. 606/92; 606/93; 606/94; 128/898

(57) ABSTRACT

A bone displacement assembly for use during a medical procedure including preparing soft bone includes a cannula and a probe that is removably inserted into the cannula. The probe includes a curved tip designed to mechanically displace soft bone and to prepare the bone tissue for subsequent treatment such as cement injection. The probe also includes a handle assembly that connects with the cannula and also includes one control device for independently rotating the distal tip and a second control device to independently translate the distal tip.

### FIELD OF THE INVENTION

The present invention relates generally to an assembly for preparing bone structures and more particularly to a probe adapted to precisely displace soft bone.

### BACKGROUND OF THE INVENTION

A number of apparatus have been developed for accessing target areas of bone or tissue within a patient. Procedures such as vertebroplasty and kyphoplasty require the insertion of an access device such as a cannula into a target area of bone to achieve access to an implantation site. In a vertebroplasty procedure, the cancellous bone of a treated vertebra is supplemented with “bone cement,” e.g., polymethylmethacrylate (PMMA) or another material, in order to provide for stabilization of the vertebral body. In a kyphoplasty procedure, an expandable device such as a balloon is inserted into the interior of the vertebra and expanded. Following removal of the expandable device, the resulting void is typically filled with bone cement to promote stabilization of the vertebral body. Vertebroplasty and kyphoplasty are desirable from the standpoint that each is minimally invasive as compared to a conventional procedures requiring surgically exposing a tissue site that is to be supplemented with bone cement.

Several procedures are known for accessing a desired site in the cancellous bone of a vertebral body, or substantially any other cancellous bone, to deliver an expandable device and/or bone cement or another suitable hard tissue implant material to stabilize, or build up, a target site as taught by U.S. Pat. No. 6,280,456, U.S. Pat. No. 6,248,110, U.S. Pat. No. 5,108,404, and U.S. Pat. No. 4,969,888, which are each incorporated herein by reference.

To gain access to a hard tissue implantation site, as described in U.S. Pat. Nos. 6,019,776 and 6,933,411, which are each incorporated herein by reference, a straight needle or cannula in combination with a stylet may be employed. As discussed therein, a stylet incorporating self-tapping threads may be utilized to obtain transpedicular access to the area of cancellous bone within a vertebral body. Once access is achieved and the stylet is removed from the cannula, bone cement may be delivered through the cannula for the purposes of filling the hard tissue implantation site.

Before cement delivery, it may be desirable to prepare the soft bone area. This preparation may be desirable in order to reduce the fragility of a fractured vertebra or diseased long bone for example and may also improve cement delivery. Several prior teaching have discussed mechanically displacing soft bone as taught by the literature, including Open Reduction of Central Compression Fractures of the Tibial Plateau, Edeland, H. G. in Acta. Orthop. Scan.; 47, 686-689, 1976. In this reference, as shown in FIG. 1, a curved probe is used to reduce the compression fracture in the knee. In particular, FIG. 1 shows an existing prior art probe 108 used for repairing a compression fracture 102. A knee joint 100 is shown including a tibia 104 and femur 106 and a femoral condyle 103 that lacks support due to a compression fracture 102 directly beneath the condyle 103 in the tibial plateau 105. The prior art curved probe 108 is carefully placed beneath the compression fracture 102 and pushed repeatedly around the cancellous bone 110 surrounding the compression fracture 102. While the surgeon pushes the prior art probe 108, cancellous bone 110 surrounding the compression fracture 102 is compacted and may form transverse bone bridges. Prior art probe 108 has a curve 112 so as to permit some “swing”, which allows the user to make minor direction changes while using probe 108.

Additionally, an article entitled, Transpedicular Fixation of Thoracolumbar Vertebral Fractures, Olerud, M. D. et al; Thorcolumbar Vertebtral Fractures, Number 227, February 1988, teaches the use of a punch with a curved distal end is rotated to reduce the fragments in a vertebral body (as shown in FIG. 2). In particular, FIG. 2 shows a an existing prior art punch 202 in relation to patient's spine 200 including vertebral bodies 204. The spine 200 has been stabilized using device 206. Punch 202 with a slight curve 206 on the distal end that is shown inserted into a vertebral body 204 to reduce the fragments of cancellous bone 210 therein. Olerud also discusses insertion of a bone paste following use of punch 202.

Notably, in both the Edeland and Olerud reference the punch or probe discussed is a relatively simple instrument that is controlled directly by the surgeon and may need to be held in position at all times to prevent instrument movement in unintended directions.

### SUMMARY OF THE INVENTION

Therefore a need has arisen for an improved system and method for precisely displacing cancellous bone in fractured or diseased bone bodies such as vertebrae or long bones.

The present disclosure presents an improved bone displacement probe removably assembled with a cannula. Once the cannula and stylet have been used to access a diseased or fractures bone site, the disclosed probe may be inserted into the cannula and used to mechanically displace soft bone, to prepare the bone tissue for subsequent cement injection. The probe may include a curved tip designed to mechanically displace the soft bone. The probe may also include a handle assembly that removably connects with the cannula and also includes at least two controls that separately and independently move the tip is desired directions. For example, one control may independently rotate the distal tip while the second control may independently translate the distal tip.

In one aspect, a bone displacement assembly is disclosed including a cannula and a probe. The cannula is adapted for percutaneously accessing a target tissue within a bone body and has a tubular elongate body and a handle. The probe has a handle assembly and an elongate body. The elongate body is adapted for slidable insertion and removal from the cannula and has a longitudinal axis and a distal tip adapted for displacing soft bone. The handle assembly is adapted for precise control of the distal tip, and has a connection portion, a tip rotation control and a tip translation control. The connection portion is adapted to removably attach to the cannula handle, the tip rotation control is adapted to rotate the distal tip about the longitudinal axis and the tip translation control is adapted to translate the distal tip along the longitudinal axis.

In another aspect, a bone displacement assembly is disclosed including a cannula, probe and a clip. The cannula is adapted for percutaneously accessing a target tissue within a bone body and includes a tubular elongate body and a handle. The probe includes a handle assembly and an elongate body. The probe elongate body is adapted for slidable insertion and removal from the cannula and includes a longitudinal axis and a distal tip, adapted for displacing soft bone. The handle assembly is adapted for precise control of the distal tip and includes a connection portion, a tip rotation control and a tip translation control. The connection portion is adapted to removably attach to the cannula handle, the tip rotation control is adapted to rotate the distal tip about the longitudinal axis and the tip translation control is adapted to translate the distal tip along the longitudinal axis. The clip is adapted to further secure the cannula handle to the handle assembly.

In yet another aspect a bone displacement system is disclosed including a cannula, a stylet, a probe and a settable implant injection delivery system. The stylet is adapted to fit within and be removed from the cannula. The probe is also adapted to fit within and be removed from the cannula and includes a handle assembly, an elongate body and a distal tip adapted for displacing soft bone. The elongate body includes a longitudinal axis. The handle assembly is adapted for precise control of the distal tip and includes a connection portion, a tip rotation control and a tip translation control. The connection portion is adapted to removably connect with the cannula, the tip rotation control is adapted to independently rotate the distal tip about the longitudinal axis and the tip translation control is adapted to independently translate the distal tip along the longitudinal axis. The settable implant injection delivery system includes a connector adapted to fluidly connect the delivery system to the cannula, to deliver a settable implant material to a target site.

In yet another aspect, a medical procedure is disclosed where a bone displacement assembly is used on a bone body. This procedure utilizes a bone displacement assembly including a cannula with an elongated tubular member and handle, as well as a probe that includes a handle assembly and a distal tip. The target tissue is penetrated with the cannula and the probe is then inserted through the cannula and into the target tissue. The probe is then fixedly attached to the cannula. The probe's distal tip may then be independently translated to displace cancellous bone and/or the distal tip may also be independently rotated to displace cancellous bone. The probe may then be removed in order for the tissue to receive further treatment.

The present disclosure includes a number of important technical advantages. One technical advantage is that the probe distal tip may be precisely moved in order to precisely displace bone. Another advantage is that this tip movement is precisely moved in independent directions through the use a separate control mechanisms, allowing a surgeon to have precise directional control the movement and placement of the distal tip and resulting cancellous bone displacement. Another advantage is that the user may release the probe during the procedure while positioned within patient, without causing unintentional bone displacement. Such release may be needed during patient imaging such as fluoroscopy, so as to locate the probe distal tip within the patient and ensure distal tip position before and during bone displacement. Due to the radiation used in most imaging devices, it is preferable that the user be outside the field of radiation and therefor temporarily release hold in probe during that time. Additional advantages will be apparent to those of skill in the art and from the figures, description and claims provided herein.

### BRIEF DESCRIPTION OF THE DRAWINGS

The invention may best be understood by reference to the following description taken in conjunction with the accompanying drawings in which:

FIG. 1 shows a prior art probe reducing a tibial fracture;

FIG. 2 shows a prior art instrument in a vertebral body;

FIG. 3 shows an assembly for accessing and displacing soft bone according to teachings of the present disclosure;

FIGS. 4A and 4B shows a probe for displacing soft bone according to the teachings of the present disclosure;

FIG. 4C shows a tip configuration for a bone displacing probe;

FIGS. 5A and 5B shows an attachment method between a cannula and probe according to the teachings of the present disclosure;

FIG. 6 shows an alternative embodiment for an attachment between a cannula and probe;

FIG. 7 shows a cannula and stylet access assembly;

FIG. 8 shows a system for accessing and displacing soft bone including a bone displacement probe according to teachings of the present disclosure;

FIG. 9 shows a system including an expandable device;

FIG. 10 shows a system for treating a vertebral body including a cement delivery system according to teachings of the present disclosure;

FIG. 11 shows a method of using the present invention in a medical procedure; and

FIGS. 12A, B and C show an attachment method between a T-handle cannula and bone displacement probe.

### DETAILED DESCRIPTION

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Last, it is to be appreciated that unless defined otherwise, all technical and scientific terms used herein have the same meaning as commonly understood by one of ordinary skill in the art to which this invention belongs. Additionally, as discussed herein the term “soft bone” and “cancellous bone” are used interchangeably and may generally refer to any non-cortical tissue within or on a bone body that may be prepared using the device and techniques of the present disclosure.

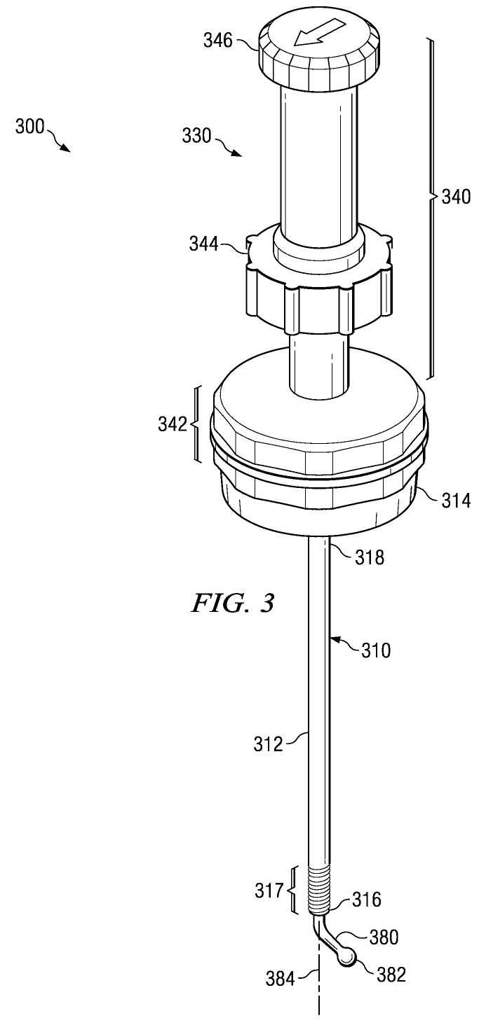

Now referring to FIG. 3, an assembly 300 for accessing and displacing cancellous bone that generally includes a cannula 310 and a probe 330 is shown. Cannula 310 includes a tubular elongate body 312 and a handle 314. Tubular body 312 defines a hollow lumen extending from a proximal end 318 to a distal end 316. Distal end 316 may terminate in a tapered or outwardly beveled distal end 317 for facilitating gripping with bone or tissue around a target site. In alternate embodiments distal end 316 may also include threads, such as self-tapping threads, for facilitating a secure and controlled access into a vertebral body. Cannula handle 314 is preferably connected with proximal end 318 of elongate body 312 and facilitates manipulation of cannula 310 and also allows communication and connection with removable stylets (not expressly shown) and/or instrumentation such as probe 330. The present embodiment shows a representative hub type handle 314; however, the present invention may be employed with any suitable handle shape or configuration such as a “T-grip” type handle, for example.

Probe 330 is sized and configured for slidable insertion into and removal from cannula 310 and comprises a handle 340 and elongate body 380 connected therewith. In the present embodiment tubular elongate body 312 has an internal diameter of approximately 0.093 inches and elongate body 380 has a cross-sectional diameter of approximately 0.080 inches. Distal tip 382 of the elongate body 380 is adapted to displace cancellous bone. In the present embodiment, distal tip 382 generally has a curvature or bend such that the end of distal tip curves away from (or is radially offset from) longitudinal axis 384. Further, distal tip 382 terminates in a slightly enlarged, blunt tip with a rounded surface. In alternate embodiments distal tip 382 may incorporate a blade or other cutting structure to allow the distal tip to more easily penetrate soft bone or to cut portions of soft bone. In the present preferred embodiment, elongate body 380 is constructed with sufficient mechanical properties to allow the curvature of distal tip 382 to straighten sufficiently to facilitate insertion through tubular elongate body 312 but also has sufficient properties to substantially regain its original curvature when the distal tip passes through or is removed from tubular elongate body 312. In one particular preferred embodiment probe elongate body 380 comprises a nitinol material.

Probe handle 340 comprises a connection portion 342 and at least two mechanisms for precisely and independently controlling distal tip 382. In the present embodiment the tip control devices include a tip rotation control 346 and a tip translation control 344. Connection portion 342 is preferably formed so as to removably connect with cannula handle 314. Tip rotation control 346 is mechanically linked with probe elongate body 380 and distal tip 382 so as to rotate tip 382 substantially about longitudinal axis 384. Tip translation control 344 is also linked by a mechanism to distal tip 382 so as to translate (or retract) distal tip 382 along longitudinal axis 384. These mechanical links are discussed in greater detail below.

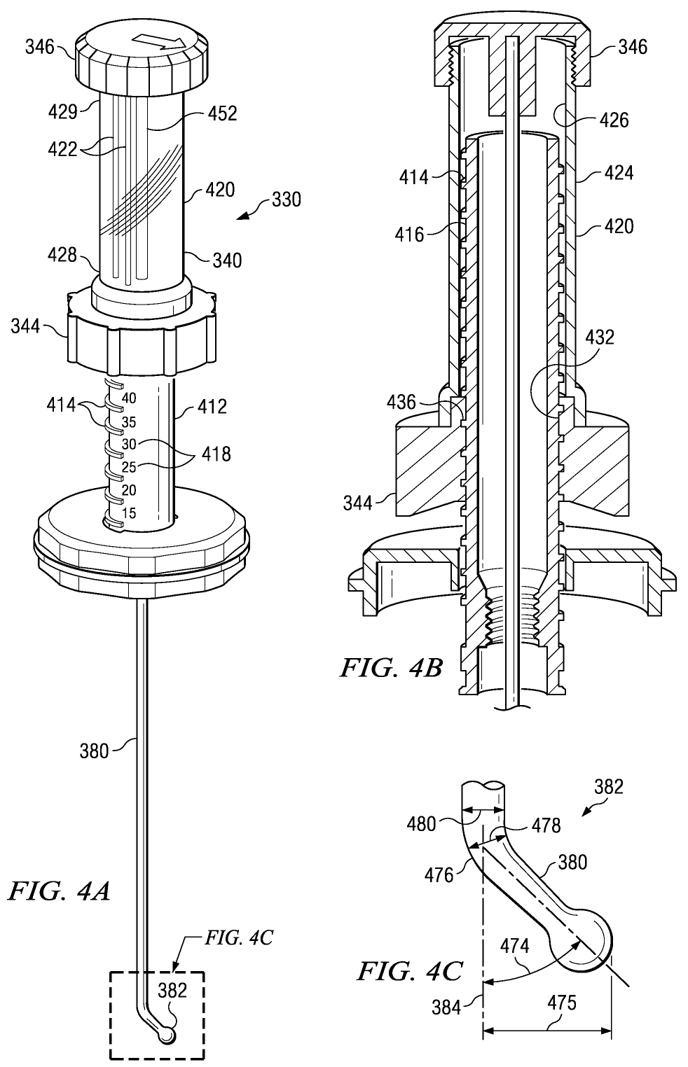

FIG. 4A shows probe 330 adapted for displacing soft bone, including handle 340 and elongate body 380. Elongate body 380 includes a distal tip 382 and proximal end 452, located within handle 340.

In the present embodiment handle 340 is assembled from two cylindrical components, an inner cylinder 412 and an outer cylinder 420, arranged concentrically. Inner cylinder 412 and outer cylinder 420 is preferably constructed from rigid materials such as polycarbonate or any other suitable material. Outer cylinder 420 is tubular and may have a generally smooth exterior surface 424 and interior surface 426 as shown in FIG. 4B. FIG. 4B shows a cross sectional view of FIG. 4A. In the present embodiment tip rotational control 346 is connected with outer cylinder proximal end 429; this connection preferably allows tip rotational control 346 to rotate independently of outer cylinder 420. Elongate body proximal end 452 is also fixedly connected with tip rotational control 346 and therefore any rotational movement of tip rotational control 346 directly rotates distal tip 382. Rotation of tip rotational control 346 may not rotate outer cylinder 420 in the present embodiment.

Outer cylinder 420 has a distal end 428 connected with a tip translational control 344. Relative translational movement between outer cylinder 420 and inner cylinder 412 is created via rotation of tip translation control 344, which subsequently advances or retracts distal tip 382, but tip translational control 344 may rotate freely with respect to outer cylinder 420.

Outer cylinder interior surface 426 may bear at least two sets of guide rails 422 protruding from interior surface 426 along a portion of the length of outer cylinder 420. The guide rails 422 retain two sets of outwardly protruding partial screw threads 414 on the exterior surface 416 of the inner cylinder 412 and the engagement of the partial screw thread 414 with the guide rails 422 prevent any relative rotation between outer cylinder 420 and inner cylinder 412. In the present embodiment partial screw threads 414 are disposed approximately 5mm apart and numerical markings 418 are printed between each screw thread 414. Numerical markings 418 provide the user with a visual indication as to the translational travel of distal tip 382.

In the present embodiment, tip translational control 344 may be connected to outer cylinder distal end 428. Tip translational control 344 is formed to provide the user with sufficient leverage to effect translation of the distal tip 382 in a precise and controlled manner. Tip translational control interior diameter 432 includes a screw thread groove 436 formed to interface with the partial screw thread 414 of the inner cylindrical component 412. When tip translational control 344 is rotated, outer cylindrical component 420 and inner cylindrical component 412 slide longitudinally relative to one another, due to the engagement of the partial screw thread 414 sliding with inner guide rails 422. This mechanism may be referred to herein as an example of a linear follower mechanism herein. As described earlier, any longitudinal movement of outer cylinder 420 will translate distal tip 382. In alternate embodiments, any suitable mechanism may be incorporated to effect the controlled, precise and independent translation of distal tip 382 within the target tissue. For example, alternate translation control embodiments may include a sliding mechanism (not shown here), whereby a tip translation control 344 may be a sliding button directly connected to a probe elongate body. Detents along a portion of inner cylindrical body may also provide fixed positions for such a sliding button to be fixed in place. In further alternate embodiments tip rotational and translational movements may not be independent of one another and may be mechanically linked to combine movements.

Tip rotation control 346 may be rigidly attached to elongate body proximal end 452 and mechanically linked with outer cylinder 420. Distal tip 382 rotation may be achieved by rotating control 346 a partial or full revolution. In the present embodiment tip rotation control 346 may rotate more than one revolution; in alternate embodiments tip rotation control may be limited to less than 360 degrees of rotation and/or may incorporate a gearing mechanism to further increase the relative precision of control between distal tip 382 and tip rotation control 346.

The user may therefore selectively rotate the distal tip 382 by rotating the tip rotation control 346. Independent from controlling tip rotation, the user may also selectively advance and retract distal tip 382 by rotating tip translation control 344. Both actions allow the user to precisely control distal tip 382, and thereby selectively and precisely displace soft bone within the target shape. Further, independent rotation and translation of distal tip 382 may preferably result in a void or cavity having an irregular shape; the irregular shape of the resulting cavity may provide a significant advantage as compared with expandable devices such as balloons, by allowing easier penetration of bone cement or other materials subsequently injected into the treated bone body.

The tip translation control 344 and the tip rotation control 346 are designed to move only when intentionally rotated. In other words, controls 344 and 346 preferably remain stationary unless intentionally moved and that rotation of tip rotation control 346 will not effect tip translation control 344 and vice versa. This also allows the user to release assembly 300 during certain times in the medical procedure without unintentional void creation or unintentional bone displacement. During a medical procedure, imaging technology such as fluoroscopy may be used to locate the distal tip 382 within the patient and a surgeon may preferably release the assembly 300 during imaging in order to be distanced from the radiation present at the time of imaging. This control may be achieved, for example, by providing sufficient friction in the fit of the control components or through detents (not shown) formed in or around the controls 344 and 346. A combination of frictional fit, material choice and lubrication may be needed to achieve sufficient friction and yet ease of movement of the tip translation control 344 and tip rotation control 346.

FIG. 4C shows a view of distal tip 382, which is preformed to assume an angle 474 with respect to elongate body longitudinal axis 384. In the present embodiment angle 474 may be approximately forty (40) degrees. In alternate embodiments, angle 474 may be in the range of five (5) degrees through ninety (90) degrees, and preferably in the range between twenty (20) degrees and seventy (70) degrees. Angle 474 may also be adjustable in alternate embodiments. In another alternate embodiment, distal tip 382 may have a preformed curve or arcuate shape at the distal tip such that the distal end is radially offset from longitudinal axis a distance 475 in the range of 0.1 inches to 1.0 inches. In other alternate embodiments distal tip 382 may be preformed with no angle 474 or offset distance 475 during insertion through a cannula (shown in previous figures) and upon allocation of heat or energy, tip 382 may bend or move to create an angle 474 or offset 475. By varying the heat or energy supplied, offset distance 475 or angle 474 may also be varied. Angle 474 or offset 475 may also be varied using mechanical linkages. Distal tip 382 materials and shape are selected so as to be sufficiently rigid to displace soft bone and yet be sufficiently flexible to bend or flex in order to fit down a cannula lumen (shown in previous figures) but to regain the preformed shape upon exiting the cannula lumen. In the present embodiment distal tip 382 has a blunt, rounded shape so as to displace soft bone but to minimize any cutting soft bone tissue. Distal tip 382 is preferably constructed from a shaped memory material so that it may better retain this preformed angle 474 even during deformation while being assembled to a cannula (not shown here) and during use within a bone body. In a particular embodiment that shape memory material comprises nitinol in its superelastic state. In alternative embodiments, distal tip 382 may be made from spring steel or shape memory alloys, shape memory polymers or thermoplastics. Distal tip 382 may partially or fully comprise of a radiopaque material so as to be viewed using radiographic imaging means such as fluoroscopy or CT in order to allow a surgeon or other user to easily locate tip 382 when inserted into a bone body. Distal tip curve 476 and cross section 478 may be varied so as to flex according to the performance needs. For example cross section 478 may be varied to allow sufficient flex to fit down a cannula lumen but to retain sufficient stiffness to displace soft bone. In the present embodiment, cross section 478 is narrower than the cross section 480 of the elongate body 380.

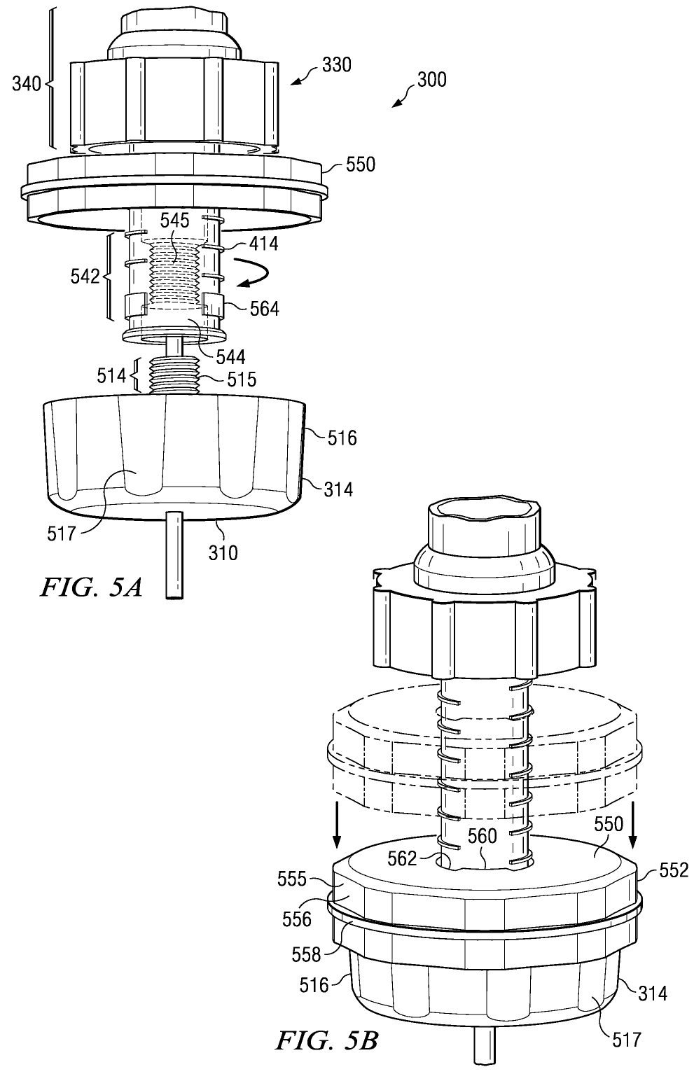

FIGS. 5A and B show the attachment between assembly 300 including a cannula 310 and probe 330. As shown, cannula handle 314 and probe handle 340 are connected so as to provide a stable base to manipulate the probe distal tip 382 (described previously).

Cannula handle 314 comprises a gripping outer portion 516 that may be circular or barrel shaped. Gripping outer portion 516 includes scallops 517 for the user to more easily grip the cannula handle 314. In addition cannula handle 314 includes an inner cylindrical threaded portion 514, which may be concentric with the gripping portion 516. This threaded portion 514 includes external threads 515 formed such that a threaded connection may be made with a threaded portion 545 of connecting portion 542 of probe 330.

Probe handle distal end 544 comprises a cylindrical threaded connecting portion 542 and a clutch plate 550. Probe threaded portion 542 and clutch plate 550 may be concentrically aligned. Probe cylindrical threaded portion 542 may include internal threads 545, outer partial threads 414 and tabs 564 and is generally formed to mate with and threadably connect with cannula inner threaded portion 514. In order to prevent this threaded connection from unintentionally rotating or loosening during use (and allowing assembly 300 to move the probe tip in an unintended manner) a secondary lock or attachment structure may be provided to prevent the threaded connection from loosening. The clutch plate 550 is one embodiment of a secondary lock that further secures the assembly's connection.

As shown in the embodiment of FIG. 5B, clutch plate 550 is generally hub shaped with an inner diameter 560 and an outer diameter 552. Clutch plate inner diameter 560 is designed to slide longitudinally over partial threads 414. Clutch 550 has at least one slot 562 in order to slide over partial threads 414 and tabs 564 so that clutch 550 may not rotate relative to threaded portion 542. Outer diameter also includes a gripping surface portion 556. Gripping surface portion 556 is generally cylindrical and comprises scallops 555 formed in the cylinder gripping portion 556 as well as a circular ridge 558 extending from gripping portion 556. Scallop 555 and circular ridge 558 may also prevent probe 330 from rolling when not in use and placed on a surface. The clutch plate 550 is designed to slide over cannula handle 314, and in combination with the cannula scallops 517 and clutch plate scallops 555, prevent the probe 330 and cannula hub 516 from rotating relative to each other, thereby securing the threaded connection there between. The ridge 558 provides a surface for sliding clutch plate 550 onto and off of cannula handle 314.

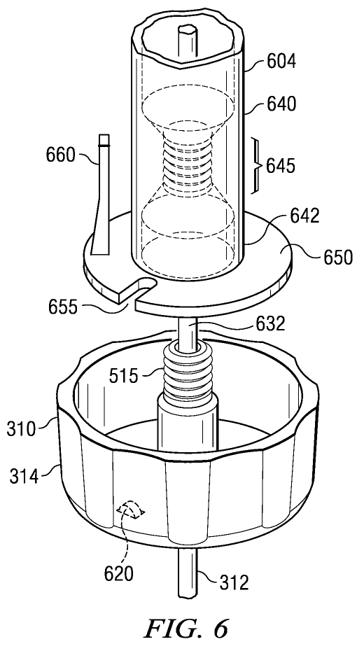

FIG. 6 shows another embodiment of attaching a cannula 310 and a portion of a probe 604. Cannula 310 comprises an elongate tubular body 312 and cannula handle 314. Probe 604 comprises an elongate body 632 and a probe handle 640. As shown, a threaded connection is formed between the cannula handle 314 and probe handle 640.as probe handle threaded portion 645 interfaces with cannula threaded portion 515.

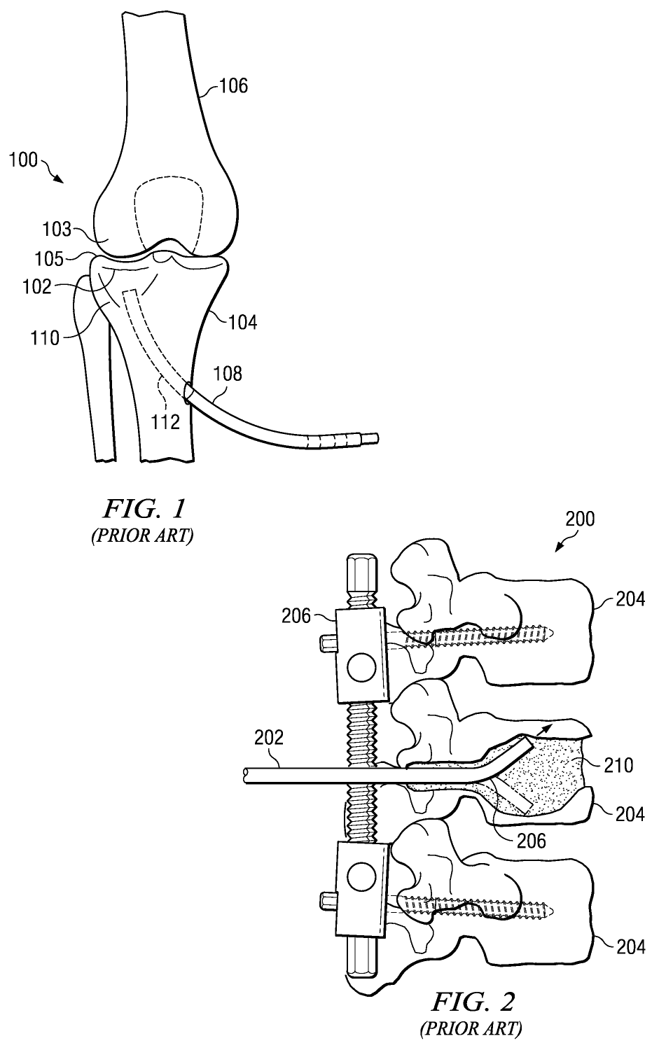

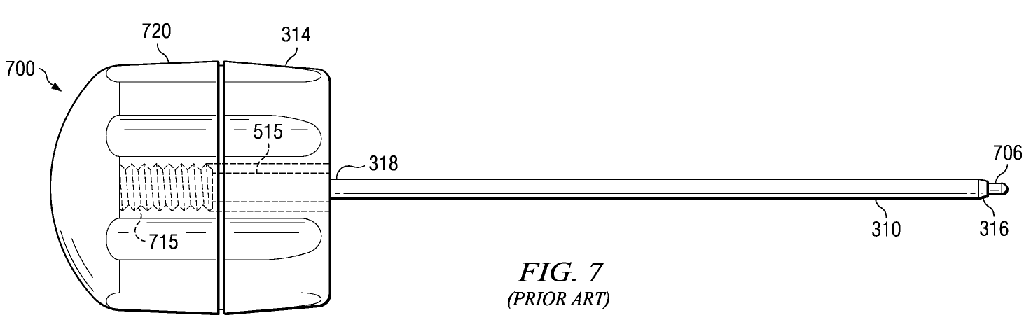

In order to prevent the threaded connection from loosening, a probe handle flange 650 is provided, radiating from probe handle distal end 642. There may be at least one slot 655 in the flange 650 formed so as to lock with at least one cannula handle key 620. As probe handle 640 is threadably connected with cannula handle 314 and while tightening the threaded connection, at least one slot 655 may be snapped into place over key 620. In the present embodiment, a short lever 660 is provided to facilitate lifting the flange area close to the slot(s) 655 during disassembly FIG. 7 shows an existing access assembly 700 that generally includes a cannula 310 and a stylet 706 operatively assembled. Stylet 706 is sized and configured for slidable insertion into and removal from cannula 310. Cannula 310 defines a hollow lumen extending from proximal end 318 to distal end 316. As shown, distal end 316 terminates in a tapered or outwardly beveled distal tip for facilitating penetration into bone or tissue of a target site. In alternate embodiments distal end may be threaded in order to facilitate placement into a treatment site. A handle 314 is provided at the proximal end 318 of cannula 310 which facilitates the user's handling and manipulation of the assembly 700. Stylet handle 720 is connected with the proximal end of stylet 706. Extending proximally from cannula handle 314 is a threaded member or connector 515 (shown in phantom in FIG. 1) for connecting with a mating connector 715 formed in stylet handle 720 and with other devices also such as a probe as discussed herein. Threaded member 515 may also be adapted for engagement with a system for the controlled injection of flowable material, such as polymethylmethacrylate based bone cement.

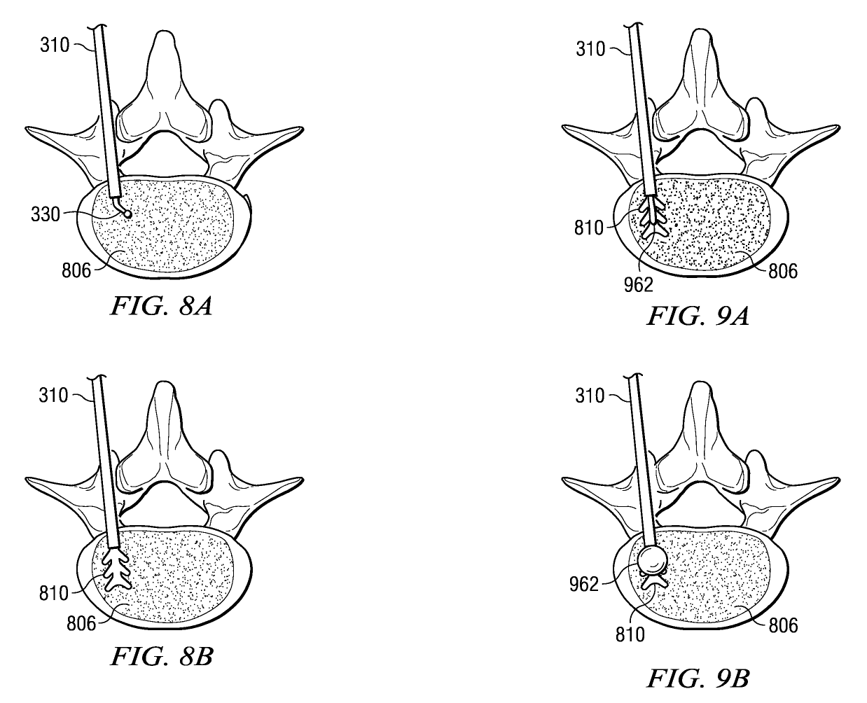

Now referring to FIGS. 8A and 8B, a depiction of a treatment system 800 being used in a medical procedure to treat a fractured or diseased vertebral body is shown. In the present embodiment, an initial transpedicular cannula placement has been achieved. In the present depiction, a bone displacement probe 330 is introduced through cannula 310 to access and displace soft or cancellous bone 806. After the selective translation and rotation of probe 330, an irregular shaped void 810 is formed therein, an example of which is shown in FIG. 8B. Void 810 is provided as an example, the geometry of the created void will vary in each procedure depending upon the anatomy of the patient, the location of diseased or fractured tissue and particular path of treatment directed by the user (the combination of probe 330 rotation and translation steps). Additionally, it should be noted that in some bone bodies void 810 may not be empty space. As probe 330 displaces or “opens” the cancellous bone in one region of the vertebral body, previously “opened” areas may be re-filled with soft bone, bone marrow, blood or other fluid contained in the bone body.

Now referring to FIG. 9A, the introduction of an expandable device 962 during a medical procedure according to teachings of the present disclosure is shown. Following placement of cannula 310 and removal of the stylet or probe 330 as described above, an expandable structure 962 is inserted through cannula 310 and placed within soft or cancellous bone 806. In some embodiments, a separate tool may first be inserted into cancellous bone 806 to create a small void in cancellous bone that is sized to facilitate initial placement of the expandable structure 962.

Following initial placement of expandable structure 962, expandable structure 962 is expanded, as shown in FIG. 9B. The expansion of structure 962 may be accomplished by introducing fluid into expandable structure 962 up to a selected pressure. As structure 962 expands, portions of soft bone 806 adjacent to the expandable structure are pushed away from expanding structure 962. Expandable structure 962 may then be collapsed and removed via cannula 310, leaving an expanded void within soft bone 806. In the embodiment shown, prior preparation of cancellous bone 806 by probe 330 may facilitate the expansion of expandable device 962 and the void created thereby. The expansion of expandable structure 962 may or may not completely envelope the void created by probe 330 depending upon the relative size and placement of probe 330's distal tip 382 and expandable structure 962.

In an alternate methodology, probe 330 may be introduced following expansion and removal of expandable structure 962. In some situations, the expansion of expandable structure 962 may form a void (not expressly shown) generally reflecting the shape of expandable structure 962 with a compacted wall of cancellous bone formed at the edges of the void. Probe 330 may then be inserted into the void and used to selectively penetrate the compacted wall of the void in order to beneficially facilitate the subsequent introduction of bone cement or another stabilizing material into the bone body outside of the void.

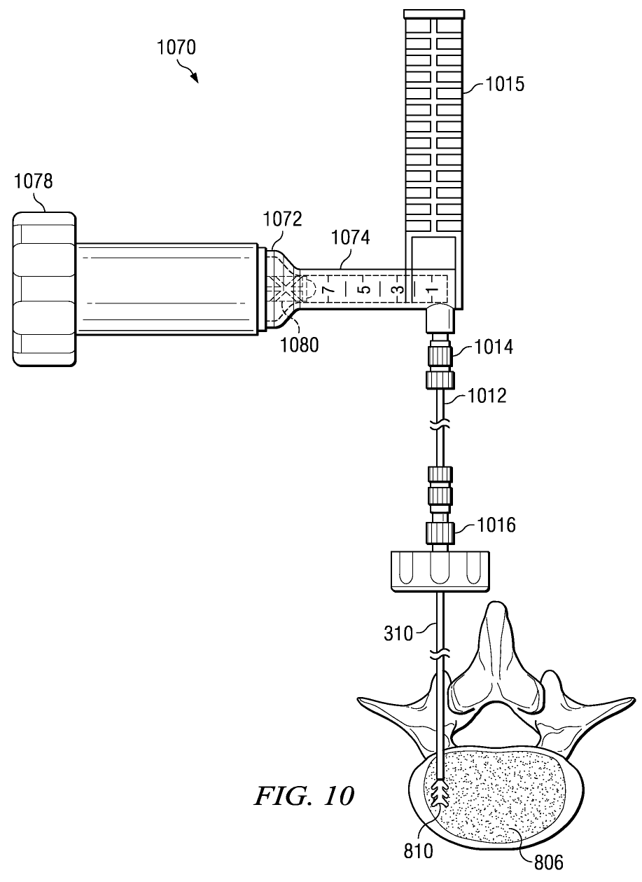

Following initial placement of cannula 310, the treatment of soft tissue 806 and/or the use of an expandable device 962, cement or another suitable filler or stabilizing material may be introduced into the treatment site or bone structure, as shown in FIG. 10. In such procedures cannula 310 is left in place at the target site. A system 1070 for the controlled injection of filler material is operatively coupled to cannula 310, so as to be in fluid communication with the cannula's lumen. System 1070 generally includes a first column 1072 and a second column 1074 which holds the filler material. A handle 1078 at the proximal end of first column 1072 is rotated in order to drive and pressurize the filler material through column 1072 and into the second column 1074. Extending distally from handle 1078 is a plunger head 1080 for forcing the filler material through the second column 1074. System 1070 is in fluid communication with cannula 310 by means of a tubing 1012 which is interconnected to system 1070 and cannula 310 by luer locks 1014 and 1016, respectively. In some embodiments, tubing 1012 may be a flexible conduit having sufficient length to remove a user's hands from a radiographic field centered at cannula 310. A handle 1015 is provided for manually handling system 1070. Once system 1070 is properly connected to cannula 310, the filler material is delivered to within the space created by the displacement probe 330 and/or expandable device 962 until a desired amount of such filler material has been injected into the treated bone body. Upon completion of the filling process, the system 1070 is disconnected from cannula 310 which may then be removed from the access site, and the wound site is treated with typical care.

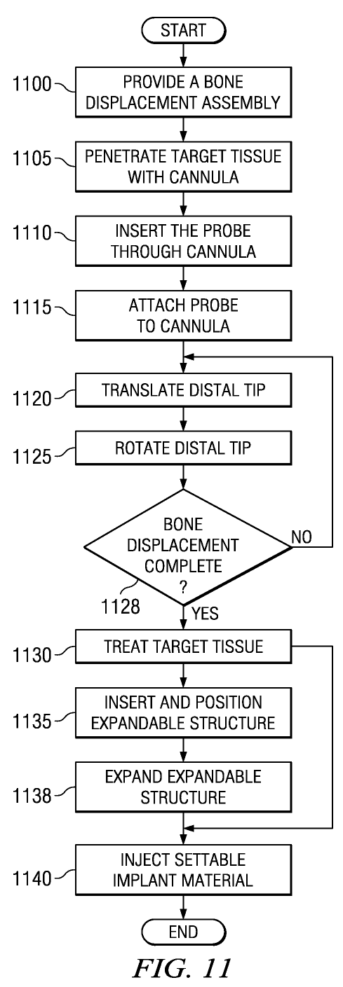

A method of performing a medical procedure using a bone displacement assembly is shown in FIG. 11. A bone displacement assembly including a cannula and a probe is first provided 1100. The cannula, typically with a stylet or obturator assembled therewith, is then inserted and target tissue is penetrated 1105. The probe is then inserted through the cannula 1110 and into the target tissue and then the probe is secured to a portion of the cannula and/or the cannula handle 1115. The probe distal tip is then independently translated to displace the cancellous bone in the target tissue 1120 and the probe distal tip may also be independently rotated to displace cancellous bone 1125. If bone displacement is then complete 1128, the target tissue is then treated 1130. If bone displacement is not complete 1128, probe distal tip may then be repeatedly translated 1120 and/or rotated 1125.

Treating the tissue may comprise inserting and positioning an expandable structure 1135 and then expanding the structure 1138. Treating the tissue may otherwise or additionally include injecting a settable implant material 1140 into the cancellous bone, as in a vertebroplasty procedure. As discussed above, in an alternative methodology, steps 1110-1125 may take place following the insertion, expansion and removal of an expandable structure.

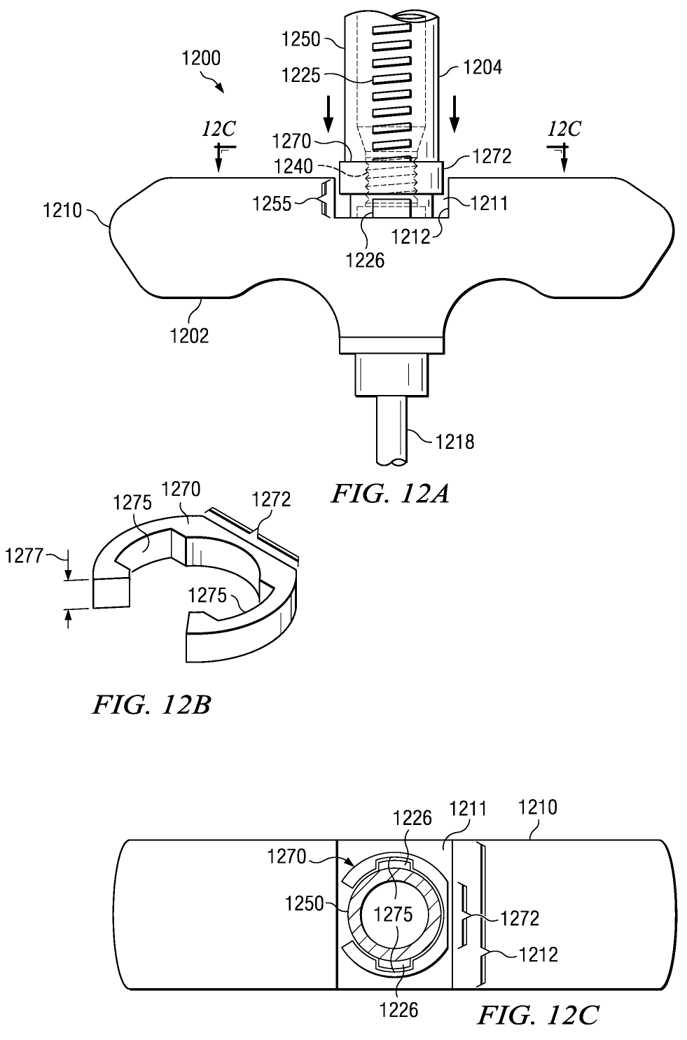

FIG. 12A shows a view of an alternative attachment assembly 1200 including a T-grip style cannula handle 1210 and probe handle 1250. Cannula 1202 comprises an elongate tubular body 1218 and cannula handle 1210, with a channel, slot or cavity 1211 within said handle 1210, substantially large enough to accommodate the probe handle 1250 and an assembled clip 1270. Probe 1204 comprises an elongate body (not shown here) and a probe handle 1250 with external partial threads 1225 and at least one tab 1226 similar in spirit to probe 330. The attachment method may include a threaded connection 1240 between cannula handle 1210 and probe handle 1250 similar in spirit to previous threaded connections described in previous figures.

In order to prevent the threaded connection 1240 from loosening, at least one clip 1270 may be used. During assembly, probe handle 1250 may be threadably connected with the cannula handle 1210 and at least one clip 1270 may be assembled to the probe handle towards distal portion 1255. Clip 1270 is formed to snap over handle distal portion 1255 and clip 1270 may include at least two slots 1275 adapted to slide longitudinally over partial threads 1225 and tab 1226. Due to the fit between clip 1270 and partial threads 1225 or tabs 1226, relative rotation between clip 1270 and probe handle 1250 is therefore restricted. Clip 1270 is formed to have thickness 1277 that will easily snap over handle distal end 1255 and then slide longitudinally so as to nest substantially within channel 1211. Clip thickness 1277 may preferably be large enough however to cover at least one partial thread 1225 and/or tab 1226 no matter where the clip 1270 location and therefore effectively prevent relative rotation between clip 1270 and probe 1250. Clip 1270 includes at least one flat portion 1272 and handle channel 1211 includes at least one flat portion 1212. FIG. 12C shows a top view of assembly 1200 including channel 1211 and channel flat portion 1212. Channel 1211 is adapted to mate with or be located in close proximity to clip flat portion 1272, so as to restrict relative rotation between clip 1270 and cannula handle 1210. Through the fit of clip 1270 within cannula handle 1210 and around probe handle 1250, probe handle 1250 and cannula handle 1210 may then not rotate relative to each other when clip 1270 is assembled and nested within handle channel 1211. In alternate embodiments channel may be shaped with alternative flat portions, notches or grooves with coinciding geometric features on a clip to prevent relative rotation between the clip and cannula handle.

Although only a few embodiments of the present invention have been described, it should be understood that the present invention may be embodied in many other specific forms without departing from the spirit or the scope of the present invention. Therefore, the present examples are to be considered as illustrative and not restrictive, and the invention is not to be limited to the details given herein, but may be modified within the scope of the appended claims.

What is claimed is:

1. A bone displacement assembly adapted for preparing soft bone comprising:
a cannula adapted for percutaneously accessing a target tissue within a bone body, having a tubular elongate body and a handle; and
a probe having a handle assembly and an elongate body;
the elongate body adapted for slidable insertion and removal from the cannula, the elongate body having a longitudinal axis and a distal tip adapted for displacing soft bone;
the handle assembly adapted for precise control of the distal tip, said handle assembly comprising a connection portion, a tip rotation control and a tip translation control;
the connection portion adapted to removably attach to the cannula handle, the tip rotation control adapted to independently rotate the distal tip substantially about the longitudinal axis and the tip translation control adapted to independently translate the distal tip substantially along the longitudinal axis.

2. The assembly of claim 1 wherein the probe handle connection portion further comprises a clutch plate formed to secure the cannula handle and probe handle assembly connection.

3. The assembly of claim 1 wherein the translation control comprises a linear follower device adapted to translate the distal tip substantially along the longitudinal axis.

4. The assembly of claim 1 wherein the probe is constructed substantially from a radiolucent material.

5. The assembly of claim 1 where the distal tip of the probe comprises a radiopaque material.

6. The assembly of claim 1 wherein the target tissue comprises cancellous bone in a vertebral body.

7. The assembly of claim 1 wherein the target tissue comprises cancellous bone in a long bone.

8. The assembly of claim 1 further comprising:
a stylet having a handle and an elongate body connected to said handle, the handle adapted for removable connection with the cannula handle and the elongate body adapted for slidable insertion and removal from the cannula, said elongate body having a distal tip, formed so as to puncture patient tissue in order to gain access to the target tissue.

9. The assembly of claim 1 wherein the distal tip of the probe comprises a blunt tip having a rounded surface.

10. The assembly of claim 1 wherein the distal tip of the probe comprises a curved shape.

11. The assembly of claim 10 wherein the curvature of the distal tip may be reduced when inserted in the tubular elongate body and returns to the curved shape when removed from the tubular elongate body.

12. The assembly of claim 1 further comprising:
a clip adapted to further secure the cannula handle to the handle assembly.

13. The assembly of claim 1 wherein the tip rotational control and tip translational control are adapted to remain stationary unless intentionally moved.

14. A soft bone displacement and treatment system comprising:
a cannula;
a stylet adapted to fit within and be removed from said cannula;
a probe having a handle assembly and an elongate body;
the probe elongate body adapted for slidable insertion and removal from the cannula, having a longitudinal axis, and a distal tip adapted for displacing soft bone;
the handle assembly adapted for precise control of the distal tip, said handle assembly having a connection portion, a tip rotation control and a tip translation control;
the connection portion adapted to removably attach to the cannula handle, the tip rotation control adapted to independently rotate the distal tip about the longitudinal axis and the tip translation control adapted to independently translate the distal tip along the longitudinal axis; and
a settable implant injection delivery system having a connector adapted to fluidly connect the delivery system to the cannula.

15. The system of claim 13 further comprising an expandable device adapted to be delivered to the target site through the cannula and expanded within the target site.

16. The system of claim 13 wherein the distal tip of the probe comprises a blunt tip having a rounded surface.

17. The assembly of claim 1 wherein the distal tip of the probe comprises a curved shape.

18. A medical procedure to be performed on a bone body comprising:
providing a bone displacement assembly including a cannula having a tubular member and a handle, and a probe having a handle assembly and an elongate body with a distal tip;
penetrating the target tissue with the cannula;
inserting the probe elongate body through the cannula and into the target tissue;
attaching the probe handle assembly to the cannula handle;
independently translating probe distal tip to displace cancellous bone;
independently rotating probe distal tip to displace cancellous bone;
withdrawing the probe elongate body; and
treating the target tissue.

19. The medical procedure of claim 18 wherein the step of treating the target tissue comprises:
inserting an expandable structure in the cannula;
positioning the expandable structure within the target tissue; and
causing the expandable structure to assume an expanded geometry.

20. The medical procedure of claim 18 wherein the step of treating the target tissue comprises injecting a settable implant material into the target tissue.

21. The medical procedure of claim 18 wherein the steps of independently translating probe distal tip and independently rotating probe distal tip creates a void having an irregular shape within the target tissue.
